(19) United States
(12) Patent Application Publication
(10) Pub. No.: US 2003/0062657 A1
(43) Pub. Date: Apr. 3, 2003

(54) On-chip membrane maker

(75) Inventors: Lalitha Parameswaran, Burlington, MA (US); Albert Young, Fishkill, NY (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Massachusetts Institute of Technology, Cambridge, MA (US)

(21) Appl. No.: 10/209,321

(22) Filed: Jul. 30, 2002

Related U.S. Application Data
(60) Provisional application No. 60/309,259, filed on Jul. 31, 2001.

(51) Int. Cl. B29D 24/00; B29D 29/08; C12N 1/00

(52) U.S. Cl. 264/553; 435/317.1

(57) ABSTRACT

An apparatus for producing membranes is disclosed, and methods for making same.

### GOVERNMENT SUPPORT

The invention was supported, in whole or in part, by grant F19628-00-C-002 from the United States Air Force. The Government has certain rights in the invention.

### RELATED APPLICATION

This application claims the benefit of U.S. Provisional Application No. 60/309,259, filed Jul. 31, 2001.

The entire teachings of the above application are incorporated herein by reference.

### BACKGROUND OF THE INVENTION

The key structural element of most biological systems is an organic membrane. This membrane is found as the outer covering of a cell and its organelles, and most sensory and regulatory proteins are embedded within, or span, the cell membrane.

Naturally-occurring cell membranes are made up of a variety of lipids, including phospholipids, glycolipids, and sterols, and mixtures of these. Membrane lipids are amphipathic, with a hydrophobic tail and a polar hydrophilic head, which spontaneously form a lipid bilayer in aqueous solutions, where the tails are situated towards the center of the bilayer, and the polar heads pointing outwards. Biological membranes are therefore referred to as lipid bilayer membranes, or bilayer lipid membranes (BLMs).

Current techniques for studying and working with membrane-bound sensory and regulatory proteins involve either using portions of natural cell membranes by patch-clamping from cells, or manually forming BLMs over small apertures. These techniques are somewhat cumbersome and do not lend themselves to automated fabrication or large scale analysis.

Synthetic membranes and filters are vital elements of microfluidic systems. Such synthetic membranes are subject to damage, e.g., rupture and clogging, during use.

The ability to generate and regenerate artificial cell membranes, and polymer membranes and filters in a microfluidic system, has both functional and economic advantages.

### SUMMARY OF THE INVENTION

Described herein is an apparatus, referred to as a “Membrane Maker” device, that is capable of generating membranes using phospholipid solutions or polymers. The apparatus can also be used to generate synthetic membranes using polymers, e.g., thermoplastics.

Also described herein are methods of making such membranes. The membranes may be provided with or without pores, using a variety of application-dependent materials. The device may also have the capability of continuously monitoring the health of the membrane during generation and usage.

In one embodiment, the invention features a method of producing an artificial cell membrane, which includes (a) dispensing a membrane lipid across an aperture, and (b) applying suction at the sides of the aperture, thereby causing the membrane lipid to thin, thereby producing an artificial cell membrane. Alternatively, instead of suction applied at the sides of the aperture, pressure can be applied to the face(s) of the aperture. The method can also include the step of determining the capacitance of the membrane, thereby measuring the thickness of the membrane. The capacitance can be determined during the application of suction or pressure, and once the desired thickness of the membrane is achieved, the suction or pressure is stabilized, thereby producing an artificial cell membrane of a desired thickness.

The membrane lipid can be a naturally-derived membrane lipid, or a synthetic membrane lipid. The membrane lipid can be a phosphatidyl choline, a phosphatidyl ethanolamine, a phosphatidyl glycerol, a phosphatidyl serine, a phosphatidyl inositol, or a sphingomyelin, or a mixture of one or more of any of the above. The membrane lipid can be a mixture of different membrane lipids, e.g., a mixture of phosphatidyl choline and phosphatidyl ethanolamine. Components of cell membranes can be included in the membrane lipid, thereby producing membranes containing incorporated cell membrane components, e.g., receptor proteins, transport proteins, ion channel proteins, antibody receptor proteins, signaling proteins, etc.

An electrical voltage can be applied across the membrane during or after membrane formation, thereby causing pores to develop in the membrane.

The invention also features a method of producing a membrane, comprising (a) dispensing a polymer in liquid form across an aperture, and (b) applying suction at the sides of the aperture, thereby causing the polymer in liquid form to thin, thereby producing a membrane. Alternatively, instead of suction applied at the sides of the aperture, pressure can be applied to the face(s) of the aperture. The method can also include the step of determining the capacitance of the membrane, thereby measuring the thickness of the membrane. The capacitance can be determined during the application of suction or pressure, and once the desired thickness of the membrane is achieved, the suction or pressure is stabilized, thereby producing an artificial cell membrane of a desired thickness.

The method can also include heating the polymer prior to the polymer being dispensed across the aperture. The method can also include application of an electrical current across the aperture during membrane formation, thereby causing pores to develop in the membrane. The polymer can be a thermoplastic.

In another aspect, the invention features an apparatus for producing an artificial cell membrane. The apparatus can comprise (a) a member with at least one aperture, and (b) at least one side channel adjacent to the aperture, through which suction can be applied across the aperture, where, when a membrane lipid is applied across the aperture, an artificial cell membrane is formed. Alternatively, instead of suction applied at the sides of the aperture, pressure can be applied to the face(s) of the aperture.

In a further aspect, the invention features an apparatus for producing a membrane, where the apparatus includes (a) a member with at least one aperture, and (b) at least one side channel adjacent to the aperture, by which suction can be applied across the aperture, where, when a polymer in liquid form is applied across the aperture, a membrane is formed.

An apparatus of the invention can further comprising at least one electrode laterally adjacent to the aperture. The apparatus can also include an instrument for determining the capacitance between the electrodes. Such electrodes can be used to determine the capacitance, and therefore the thickness, of the membrane. The electrodes can also be used to apply an electrical voltage across the aperture during and/or after membrane formation, thereby causing pores to develop in the membrane. The electrodes can also be used to measure ion current flow and membrane capacitance across the membrane, and through openings (e.g., ion channels, pores), and the movement of charged particles through the membrane.

The membrane lipid can be a naturally-derived membrane lipid, or a synthetic membrane lipid. The membrane lipid can be phospholipid, a glycolipid, or a sterol. The membrane lipid can be phosphatidyl choline, or phosphatidyl ethanolamine. The membrane lipid can be isolated from natural sources, or synthesized chemically. The membrane lipid used to form the membrane can also be a mixture of membrane lipids, such as a mixture of phosphatidyl choline and phosphatidyl ethanolamine. Components of cell membranes (e.g., receptor proteins, antibody receptor proteins, signaling proteins, transport proteins, ion channel proteins, ion pumps, nutrient transporters, and other membrane transport systems) can also be included in the membrane lipid.

### BRIEF DESCRIPTION OF THE DRAWINGS

FIGS. 1A and 1B are a set of two diagrams illustrating one embodiment of the “Membrane Maker”. FIG. 1A shows the apparatus as having an aperture of greater than 100 μm, with two side channels, and fluid inlet located at the top, and two electrodes. FIG. 1B shows the a membrane being made. A droplet of membrane lipid (e.g., phospholipid, a glycolipid, or a sterol) inters via the fluid inlet, attaches to the aperture rim, and suction pressure is applied to aid in BLM formation.

FIG. 2 is a chart outlining examples of uses for regenerable bilayer lipid membranes (BLMs).

FIG. 3 is a schematic cross section of an example membrane maker and an outline of membrane formation.

FIG. 4 is a schematic and chart outlining an example of a technical approach for a simplified multi-part BLM support structure.

FIG. 5 is a schematic and chart outlining basic BLM formation mechanics.

FIG. 6 is a chart of BLM development.

FIG. 7 is a schematic and chart outlining a preliminary aperture with a lipid membrane.

FIG. 8A is a schematic of a basic membrane-maker.

FIG. 8B is a schematic of an advanced membrane-maker.

FIGS. 9A-9D are illustrations of a membrane formation procedure. FIG. 9A depicts the membrane material in a solid state. FIG. 9B depicts heating of the membrane material to cause it to flow; the application of flanking fluid pressure to shape the membrane; and the removal of the heat by turning off the heat source to solidify the membrane. FIG. 9C depicts the opening of filtration pores via electroporation, and monitoring of the capacitance/ionic flow through the filter. FIG. 9D depicts the removal of the damaged membrane filter.

FIG. 10 is an example schematic of the use of an automated BLM device to form complex membrane assemblies. The outlined example illustrates coupling of B-cell receptor activation with the IP₃ pathway.

FIG. 11 is a schematic and chart example of a filter-generation procedure.

FIG. 12 is an illustration of electroporation electrodes.

FIG. 13 is a schematic and chart of an enhanced membrane maker.

FIG. 14 is a chart of examples of technical challenges for regenerable filters.

FIG. 15 is a schematic of T³ design features depicting regenerable BLM hardware and electronically addressable transcription.

FIG. 16 is a schematic graph depicting engineering bioelectromechanical systems (BEMS). On the x-axis is the number of biological elements. On the y-axis is the number of controllable degrees of freedom.

FIG. 17 is a schematic and chart of bio/silicon integration from a living biological cell to a non-living biosystem.

FIG. 18 is a schematic of bilayer lipid membranes (BLMs) as a key foundation element in artificial biosystems.

FIG. 19 is a schematic and chart of bilayer lipid membrane basic principles.

FIG. 20 is a schematic and chart of a technical example for a multi-part BLM support structure.

FIG. 21 is a schematic and chart of micromachined apertures.

FIG. 22 is an illustration and chart of observed membrane thinning.

FIG. 23 show graphs and charts of capacitance measurements of BLM during formation. Test parameters include aperture diameter, difference lipids, solvents, concentrations, temperature and aqueous solutions.

FIG. 24 show graphs illustrating capacitance measurements of BLM during formation.

FIG. 25 is a chart of accomplishments.

FIG. 26 is a schematic and a chart of vesicle (also referred to as liposome) generation using a membrane maker.

FIG. 27 is an illustration of field-assisted vesicle generation.

FIG. 28 is a schematic and chart of an example of an advanced membrane maker.

FIG. 29 is a chart of an advanced membrane maker usage.

FIG. 30 is a chart of the versatility of an advanced membrane maker.

FIG. 31 is a schematic timeline for artificial biosystems.

FIG. 32A is a diagram of a typical bilayer vesicle with membrane-bound proteins and encapsulated biomolecules.

FIG. 32B is a schematic diagram of an example apparatus and procedure used to generate vesicles from dried lipid films on a conductive substrate. Electrodes can be metal, for example Pt or Au, or alternatively, ITO.

FIG. 33 is a schematic diagram for micrographs FIGS. 33A-D, which are illustrations of phase contrast micrographs of vesicles generated from rehydrated dried lipid films.

FIG. 34 is a schematic and chart summarizing examples of a membrane maker as an enabling technology for various applications.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

A description of preferred embodiments of the invention follows.

In one embodiment, the invention comprises an apparatus, referred to as a “Membrane Maker”, that is capable of generating membranes. The membranes can be artificial (e.g., simulated) cell membranes, and can be made using phospholipid solutions. The membranes can also be synthetic membranes, and can be formed out of polymers, e.g., thermoplastics. In another embodiment, the device is also capable of continuously monitoring the health and integrity of the membrane during generation and usage. “Lipid bilayer membrane” and “bilayer lipid membrane” are used interchangeably herein.

In another embodiment, the invention comprises methods of making such membranes. The membranes may be with or without pores, and can be made using a variety of application-dependent materials.

The “Membrane Maker” can be used to generate, monitor and regenerate single membranes or arrays of membranes in micromachined substrates, all in an automated fashion. In one variation of the device, the membrane material is a phospholipid, essentially identical to that found in natural cell membranes; this membrane can serve as a substrate for proteins that have been produced via transcription/translation of nucleic acids, or other means. In another variation, the device can be used to form membranes composed of alternative polymers, such as thermoplastic-based materials that are suitable for use in fluid filtration.

In one embodiment, the Membrane Maker includes a member in a single layer, with an aperture in the member. Such an embodiment is shown in FIG. 7. The material which is to form the membrane is placed on the aperture, and allowed to spread across it to span the aperture. Pressure, either in the form of positive air or fluid pressure, can be applied to the surface of the membrane to cause it to thin to the desired thickness. Alternatively, negative pressure (e.g., a vacuum) can be applied at the outer edges of the membrane to accomplish the same result.

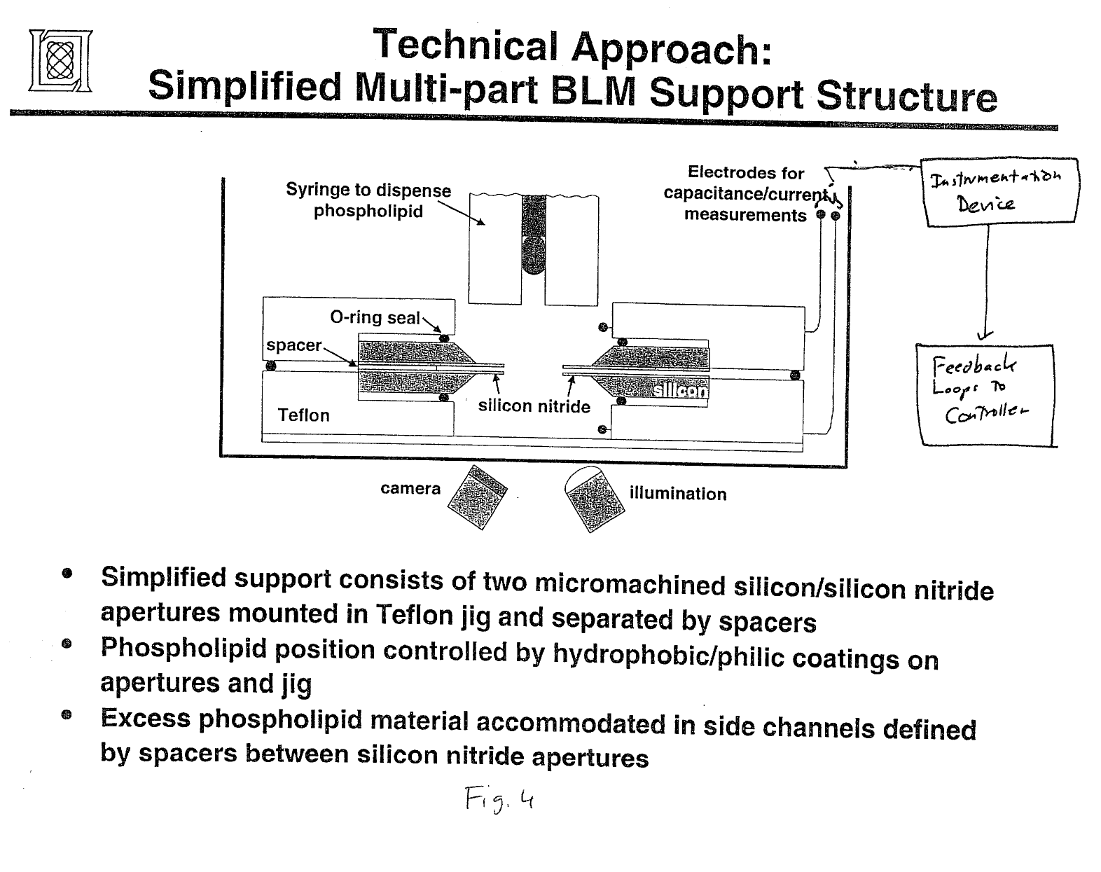

In another embodiment, the Membrane Maker includes a two-layer member, or two members placed laterally adjacent to each other, with the aperture located through both layers or both members. In this situation, “aperture” is intended to include the pair of apertures, that is, the set of two apertures, one occurring in each layer. Such an embodiment is shown in FIG. 4. The material which is to form the membrane is placed on the pair of apertures, either applied from one side of one aperture, or from between the two layers of the member(s). The material is allowed to spread across and span the aperture(s). Pressure, either in the form of positive air or fluid pressure, can be applied to the surface of the membrane to cause it to thin to the desired thickness. Alternatively, negative pressure (e.g., a vacuum) can be applied at the outer edges of the membrane to accomplish the same result.

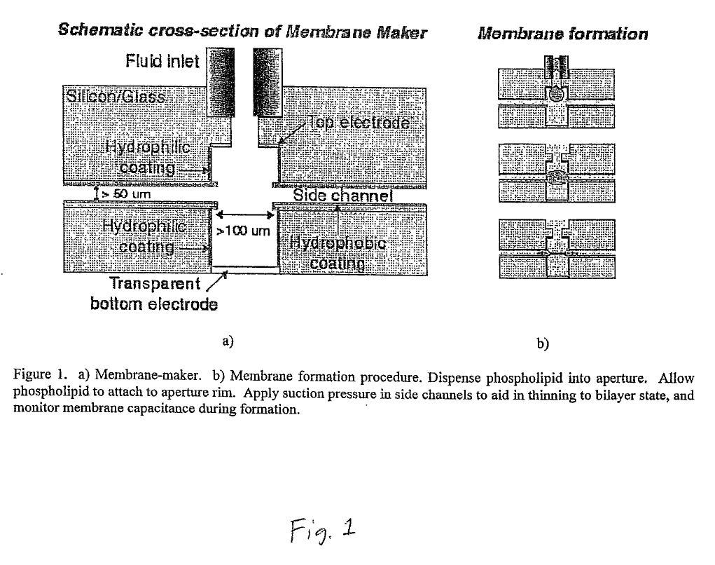

FIGS. 1A and 1B illustrate one embodiment of the membrane-maker apparatus. In general, the device has different sets of fluid channels, one to accommodate membrane material, and other(s) to hold fluid to be used for shaping the membrane, or fluid to be passed through the membrane, or suction/pressure to be applied to the membrane. The surfaces of the fluid channels can be treated to impart hyrdophobic or hydrophilic properties, to control the location and flow of fluids in the channels.

The membrane is formed within a set of at least one aperture. The aperture can be composed of a suspended silicon nitride film with a hole formed in it. The silicon nitride film can be suspended over a silicon substrate micromachined to have an opening to allow through-flow between the two sides of the aperture. If there are two apertures, they can be separated by an inert spacer (which can be composed of PDMS (polydimethyl siloxane), for example), forming a channel which can accommodate excess material not used to form the membrane.

The artificial cell membrane is formed via the natural tendency of the phospholipid to self-assemble into a bilayer state, aided by fluid pressure shaping, e.g., suction pressure applied to remove excess lipid material. The phospholipid is dispensed into the aperture and thins to a bilayer state by initiation of contact of opposing oriented monolayers through thermal, mechanical, and/or electrocompressive forces. Application of suction or pressure is designed to accelerate thinning and self-assembly of the lipid. Membrane thickness is monitored via capacitance measurements or by optical means. Measurements are fed back to a pressure controller for closed loop feedback control of the membrane thickness.

The membrane lipid can be a naturally-derived membrane lipid, or a synthetic membrane lipid. The membrane lipid can be phospholipid, a glycolipid, or a sterol. The membrane lipid can be a phosphatidyl choline, a phosphatidyl ethanolamine, a phosphatidyl glycerol, a phosphatidyl serine, a phosphatidyl inositol, a sphingomyelin, a lecithin, a phosphorylcholine, etc., or a mixture of one or more of any of the above. The classes of membrane lipids that self-assemble into bilayers are well-known in the art, and many are available commercially.

In general, any long-tailed lipid capable of self-assembly can be used in the invention to produce artificial BLMs. The lipid can have a tail of about four carbons to about 24 carbons or more. Preferably, the lipid has a tail of about 12 carbons to about 18 carbons. The “lipid” used to produce BLMs can be a mixture of such lipids, and the different lipids can have tails of approximately the same length, of different lengths, e.g., long- and short-tailed lipids can be combined.

The membrane lipid can be isolated from natural sources, or synthesized chemically. The membrane lipid used to form the membrane can also be a mixture of membrane lipids, such as a mixture of phosphatidyl choline and phosphatidyl ethanolamine. Components of cell membranes (e.g., receptor proteins, antibody receptor proteins, G protein-coupled receptors, signaling proteins, transport proteins, ion channel proteins, ion pumps, nutrient transporters, and other membrane transport systems) can also be included in the membrane lipid.

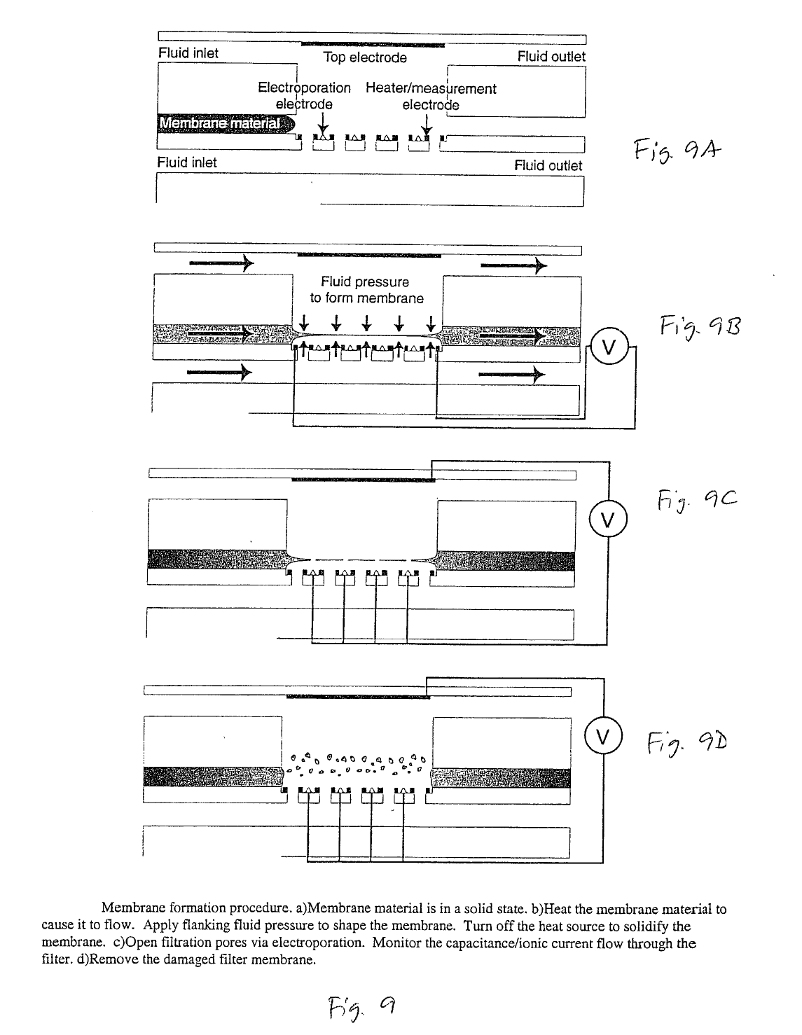

The synthetic membrane is formed via fluid pressure shaping. Membrane material is allowed to flow through a lateral channel, and is shaped via fluid pressure applied from above and below. Membrane thickness is monitored via capacitance measurements or by optical means, and measurements are fed back to the fluid shaping pressure controller, for closed loop feedback control of the membrane thickness (see, e.g., FIGS. 1 and 9).

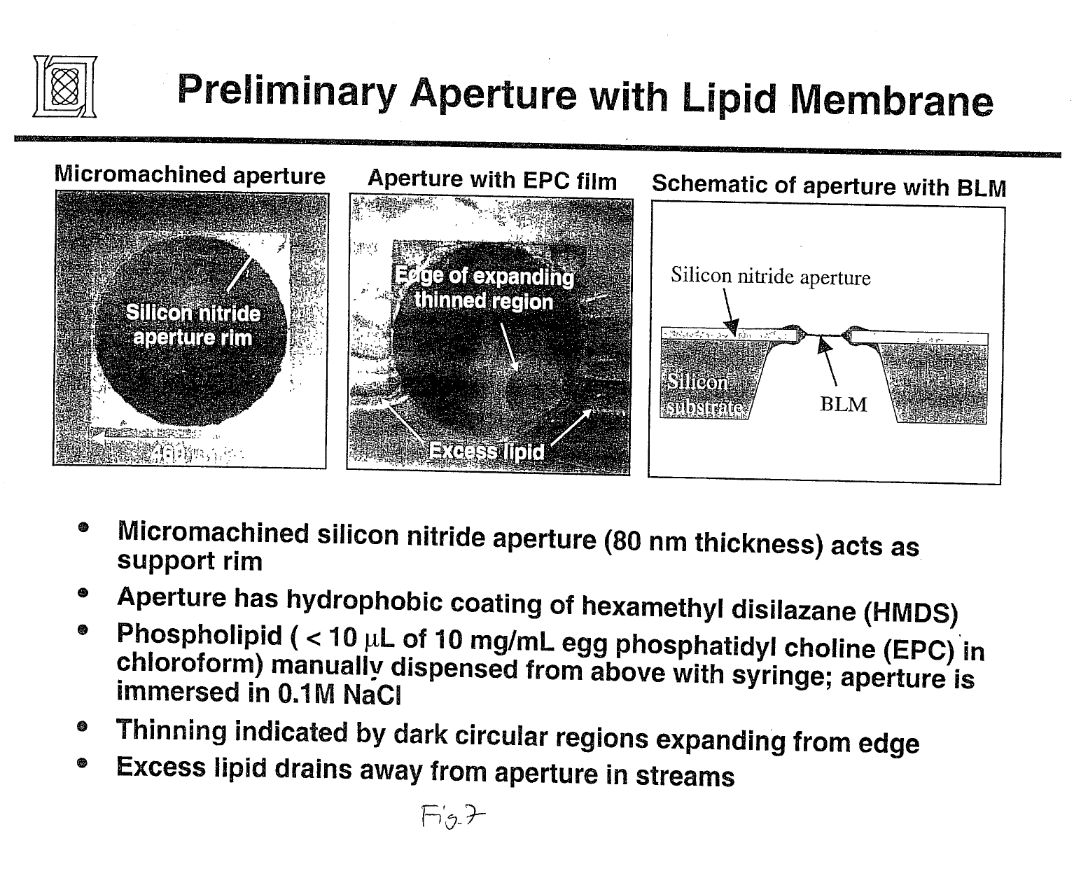

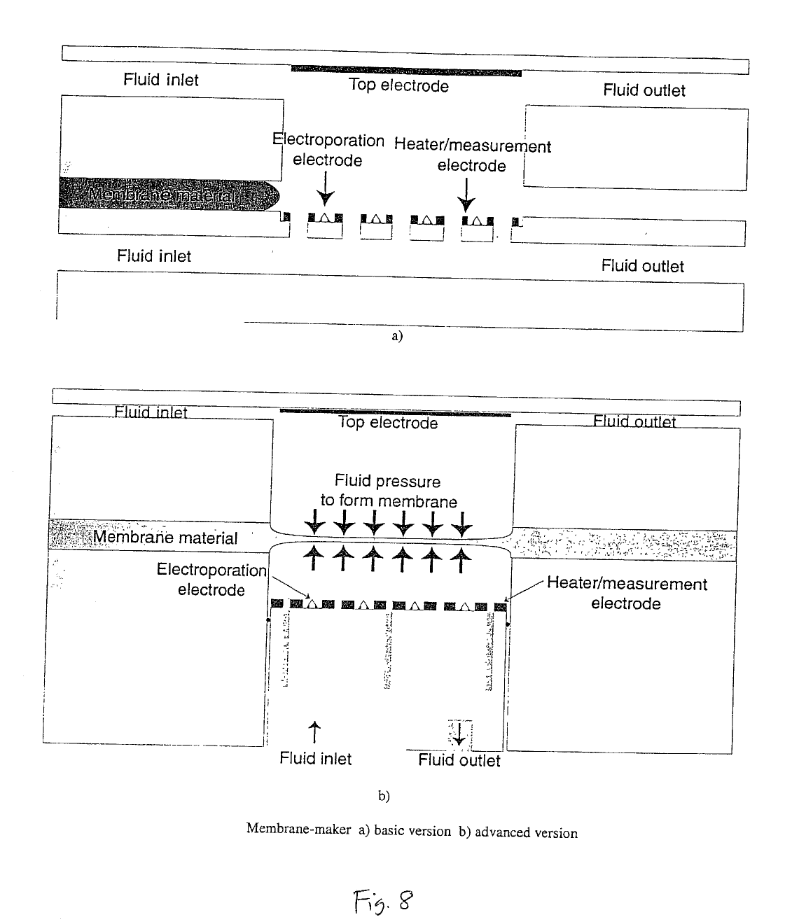

The device can have a plurality of electrodes (see, e.g., FIGS. 8A and 8B) which can serve several functions. They can be used to monitor the capacitance of the fluid-membrane system as the membrane is being formed, to control membrane thickness. They can also be used to monitor ionic current flow through the membrane, during pore formation to control pore size, as well as during usage to monitor the membrane for damage, clogging or rupture. The electrodes can be connected through feedback loops to the fluid channel valves, for control of membrane generation and regeneration.

The bottom electrode can consist of a series of individually addressable electrodes, which can take the form of metal cones. The shape of the electrodes can be determined by the required currents for electroporation. Individual addressing of each electrode enables variations in pore distribution and arrangement. The bottom electrode arrays can also be used to initiate the release of reagents in the process of incorporating membrane-bound receptors into bilayer lipid membranes (BLMs) as described in the applications section.

The bottom electrode fluid channels can be evenly distributed through the bottom electrode. They can be interdigitated sets of channels, which will allow the fluid pressure to be varied across the diameter of the membrane for shape control (e.g., corrugations). The bottom electrode position with respect to the membrane fluid channel can be adjusted, to vary the electric field applied across the membrane and thus the pore size.

Pores can be formed via an electronic process, in which a current is passed from the lower electrode through the membrane at the desired pore location to the upper electrode. Current flow is maintained until a pore of the desired size is formed. Pore size can be monitored optically or via ion current measurements, and is also controlled in a closed loop feedback mode.

FIG. 9 shows a representative process used to form, use and remove a membrane. This process applies in the case of a thermoplastic membrane to be used as a size exclusion filter. The device contains the thermoplastic to be used in solid form in the membrane fluid side channels (FIG. 9A). Heat is applied to melt the material after which it is flowed through the membrane area (FIG. 9B). At this point fluid flanking pressure is applied to shape the membrane, during which time the capacitance of the fluid-plastic system is monitored by instrument until the desired thickness is reached. Heat is removed, allowing the membrane material to solidify. Filter pores are formed via electroporation (FIG. 9C). The filter membrane is now ready for use, during which time ionic current flow or capacitance between the electrodes is monitored by instrument to check for membrane damage. If a change in current or capacitance signaling a rupture or clog is detected, the membrane can be removed in one of several ways, e.g., applying fluid pressure and/or large currents between the electrodes to break up the entire membrane and sweep out the fragments, or applying heat and fluid pressure to remelt the membrane (FIG. 9D).

In one embodiment, the thermoplastic to be used is a low-melting temperature material and is immiscible in the surrounding flanking fluid. It is also able to undergo repeated liquid-solid transitions without degradation (i.e., granulation), and have good chemical resistance, e.g., polyethylene and polyvinylidene difluoride (PVDF).

Automated generation of BLMs, as described herein, allows the maintenance of BLM stability and integrity without maintenance of any cellular infrastructure, and without cumbersome techniques such as the patch clamp method. Such membranes can be used in ion channel sensing systems, in DNA sequences and sensors, and in proteomics studies and the design and synthesis of proteins. Such membranes can also be arrayed on a chip, creating on-chip versions of cell-based sensors. Such arrays can be used to classify and assay agents via the effect on cellular receptions, and in improved pathagen characterization.

The arrays can also be used to create arrays of “artificial cells” on a chip or wafer. Such arrays can incorporate ion-channel biosensors, cellular receptions, signalling molecules, and/or transport proteins.

In another embodiment of the membrane-maker apparatus, the meniscus-like ring around the edge of the BLM could be removed using a membrane-transfer technique, where the BLM is blown up like a soap bubble and then makes contact with a receiving aperture. This can be done by contacting a liposome with the aperture, e.g., as shown in FIG. 26. This contact process serves to transfer only the thinned self-assembled bilayer portion of the membrane and eliminates the “Plateau-Gibbs” region of excess lipid material surrounding the thinned region. This could be important for high-performance applications, for example where parasitic capacitances are important.

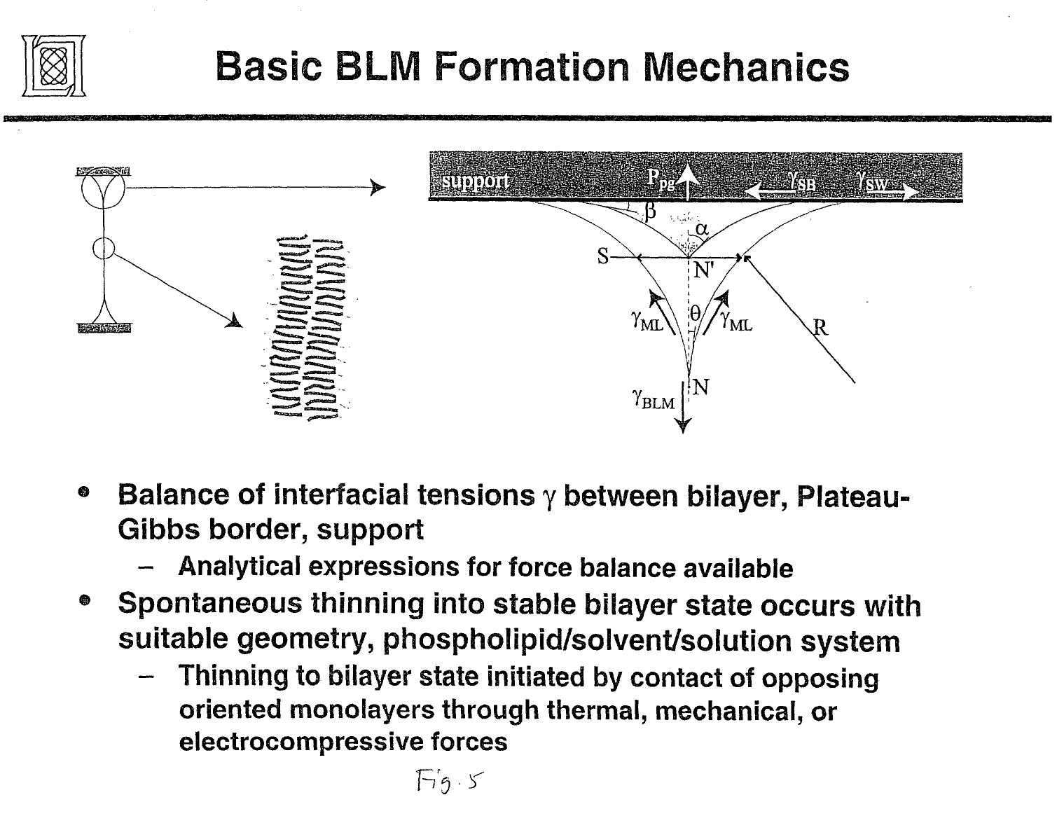

The meniscus-like ring of excess material is known as the “Plateau-Gibbs” region. The mathematical relationships between the interfacial tensions (y) between the bilayer, the Plateau-Gibbs border, and the support are shown in FIG. 5.

The analytical expressions for geometry of the stable Plateau-Gibbs border region requires interfacial tension data. Experimentally-determined interfacial tensions are available for a number of phospholipid/solvent/solution systems. The theoretical basis for thermodynamics of membrane formation using free energy is also known.

The membrane maker can be used to generate, monitor, and regenerate polymer-based size exclusion filters, and these processes can be automated. The filters can be tailored for specific purposes in their thickness, pore size and pore spacing. Size fractionators can be formed in a tailored fashion by using a series of membrane-makers and reducing the pore size and spacing is successive membranes. The device may also be used to make valves on-demand, by creating a membrane when fluid blocking is required and destroying it when through-flow is desired.

The general membrane-maker geometry can be used to form a device which is capable of forming bilayer lipid membranes (BLMs) in an automated fashion as well, for example by introducing a phospholipid bilayer in an appropriate solvent into the membrane-material channel. This is useful since BLMs suffer from manual assembly procedures and have irreproducibility and stability problems. BLM status can be monitored electrically as described above and BLM regeneration could automatically occur upon detection of membrane rupture. For example, one can use pressure feedback control of the fluid channels to control the thickness and lateral dimensions of the phospholipid layer. The sensing method can be optical or electrical.

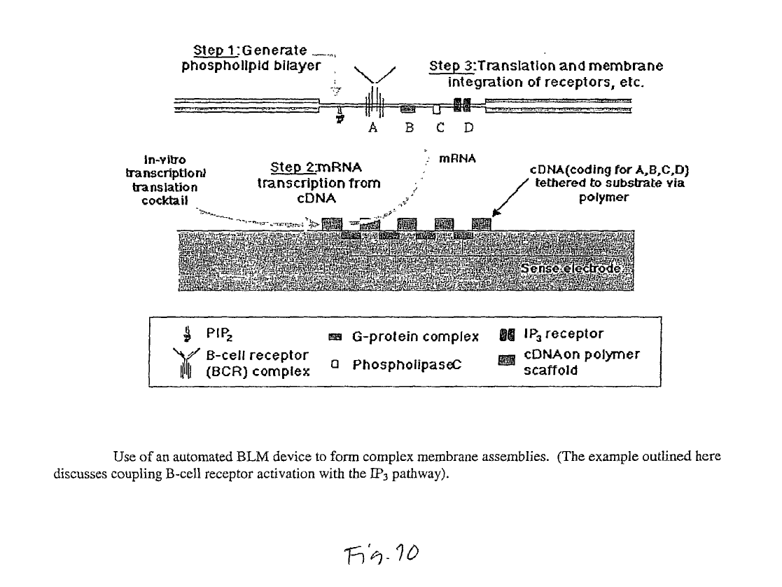

Once can use these automated BLM fabrication techniques in conjunction with in vitro translation or in vitro transcription/translation systems to reconstitute complex membrane-bound receptors and more complex molecular systems. As shown in FIG. 10, the basic process can be distilled to a three-step procedure. In step one, generation of the phospholipid bilayer takes place, much as described above. Additional components can be added to the phospholipid mixture to improve the process. In step two, in vitro transcription/translation mixtures are brought into the device. The device features pretethered pieces of cDNA coding for the proteins of interest, which are then transcribed into mRNA. These pieces of DNA can be directly tethered to the substrate (e.g., a gel matrix) in patterns or can be coupled into the substrate. In the embodiment shown in FIG. 10, the cDNA pads are designed to be close to the BLM, so that diffusion of mRNA to the membrane can take place and translation of the mRNA into the desired proteins can occur. If the correct materials are provided, membrane integration will also occur. A number of proteins that aid translocation are known, e.g., signal recognition particle (SRP) receptor, Sec61p complex, and translocation chain-associating membrane (TRAM) protein.

If electrodes are incorporated into the device, then they can be used to draw in reagents to specific sites, thus giving one control of which specific proteins are expressed from a larger array. Implementation in microfluidics would enable large arrays of both cDNAs within a device site, as well as large arrays of device sites to be developed. This would enable simultaneous analysis of many different complex molecular combinations to better understand mechanistic processes involved. This type of system also removes extraneous interferents found in cell-based systems. Rigorous physical models of these systems can then be developed, and subjected to mathematical analyses, thus changing the way biological science is performed. Additional power in this type of device comes from the fact that both sides of the membrane (and associated membrane-bound proteins) are accessible with independent fluid flows, thus enabling one to control the environment on both sides of the membrane independently. This is a powerful tool not easily available to the biological reasearcher today.

The method and apparatus described herein can be used in conjunction a system for implementing in vitro transcription and translation systems, which are used to express proteins encoded by specific sequences of DNA or RNA. In such a system, the starting nucleic acid material is tethered or localized to specific location in a solid support format, e.g., is tethered to a gel or solid matrix, magnetic bead, or chip. Introduction of an in vitro transcription/translation cocktail (e.g., predominantly rabbit reticulocyte lysate, optionally supplemented with canine pancreatic microsomes) allows mRNA transcription to proceed. Such a system enables precisely controlled localization and addressability of a desired subset of the proteins produced, and open up a wide range of applications such as array-based protein screening and analysis on a large scale.

Such in vitro transcription/translation systems are commercially available and are commonly used to express in vitro the protein encoded by a given DNA or RNA sequence. These systems are advantageous because DNA is more stable than RNA or protein, and because they eliminate the possibility of cell-based intereference.

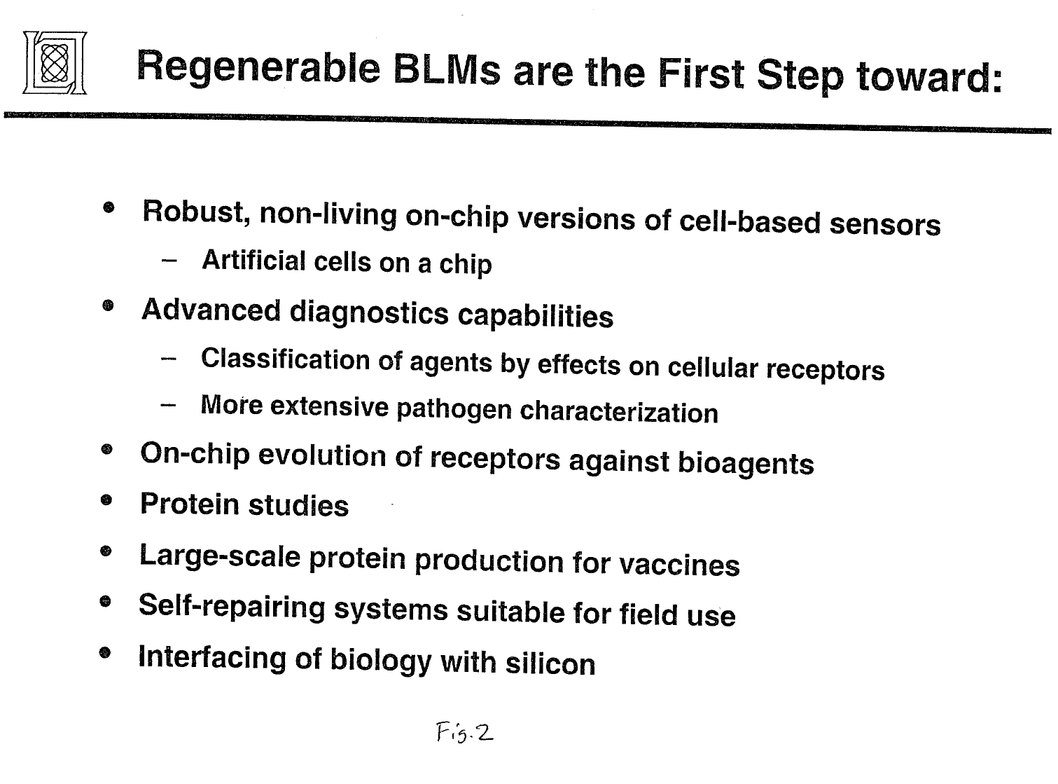

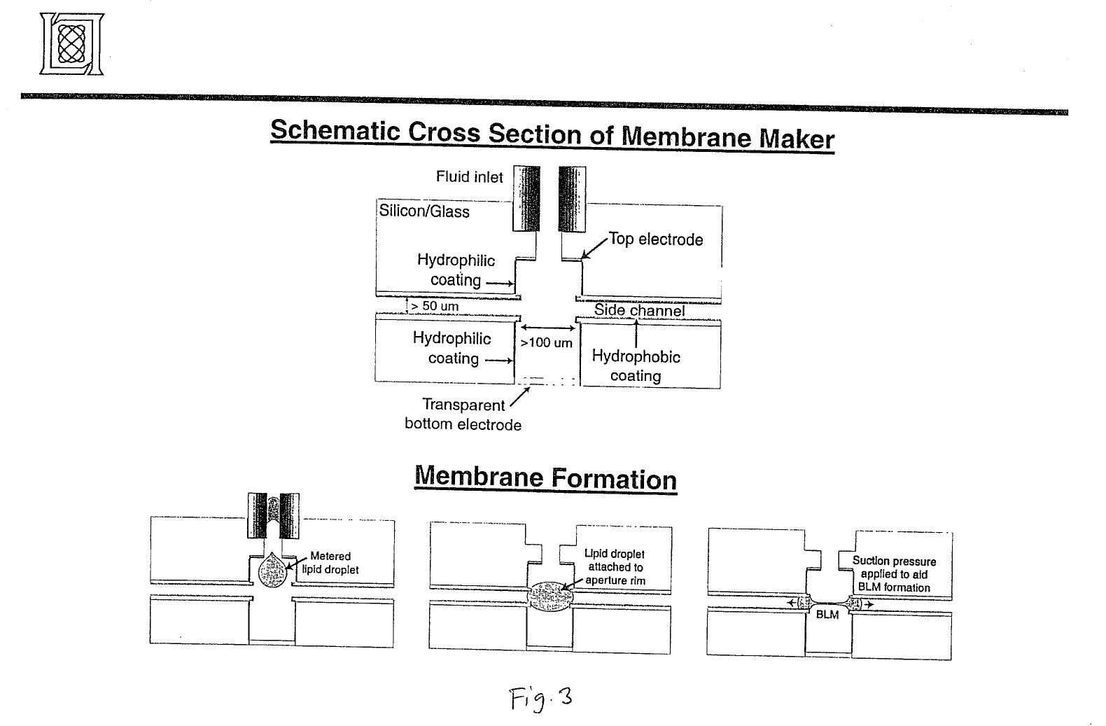

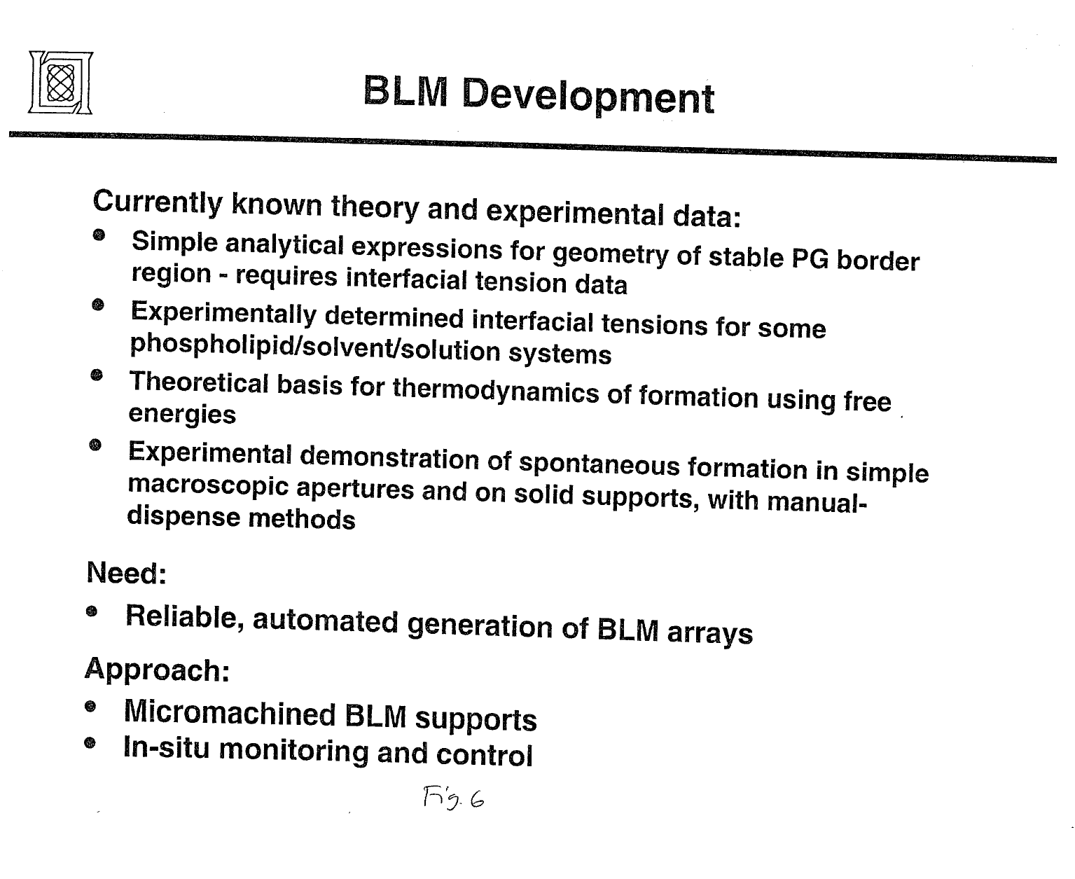

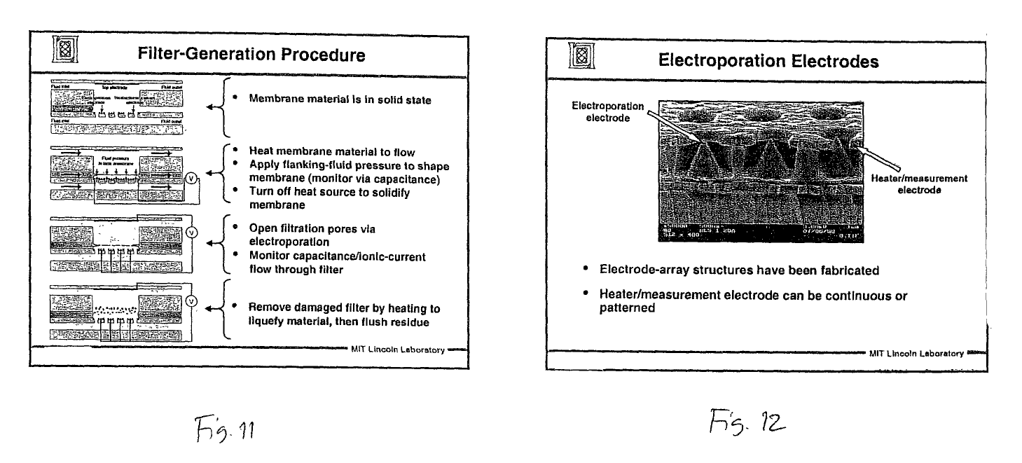

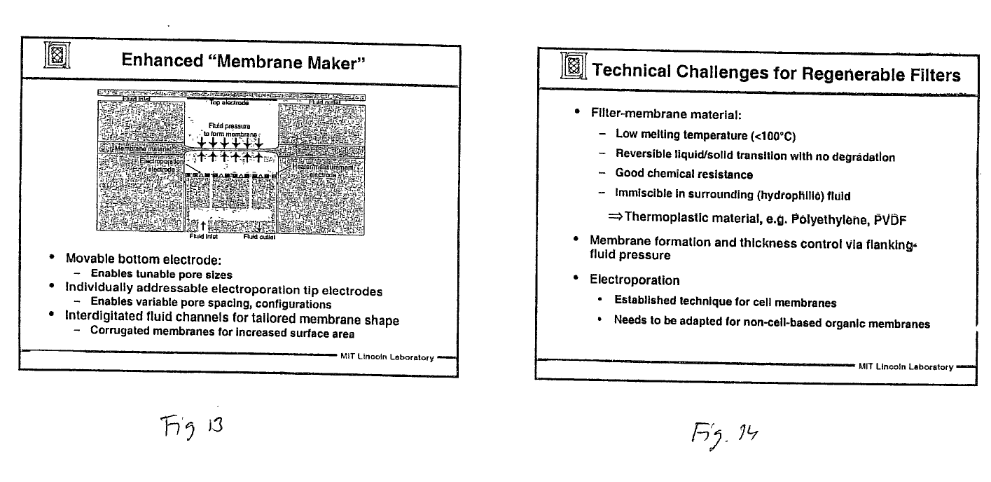

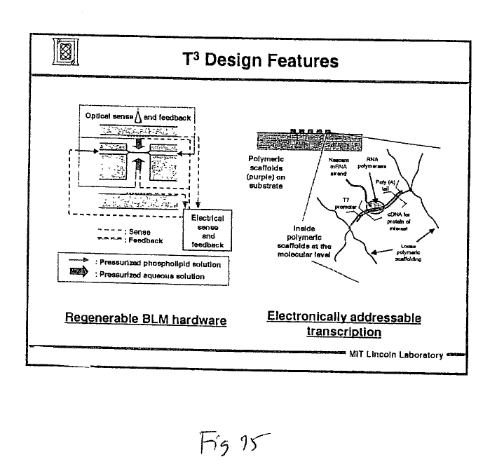

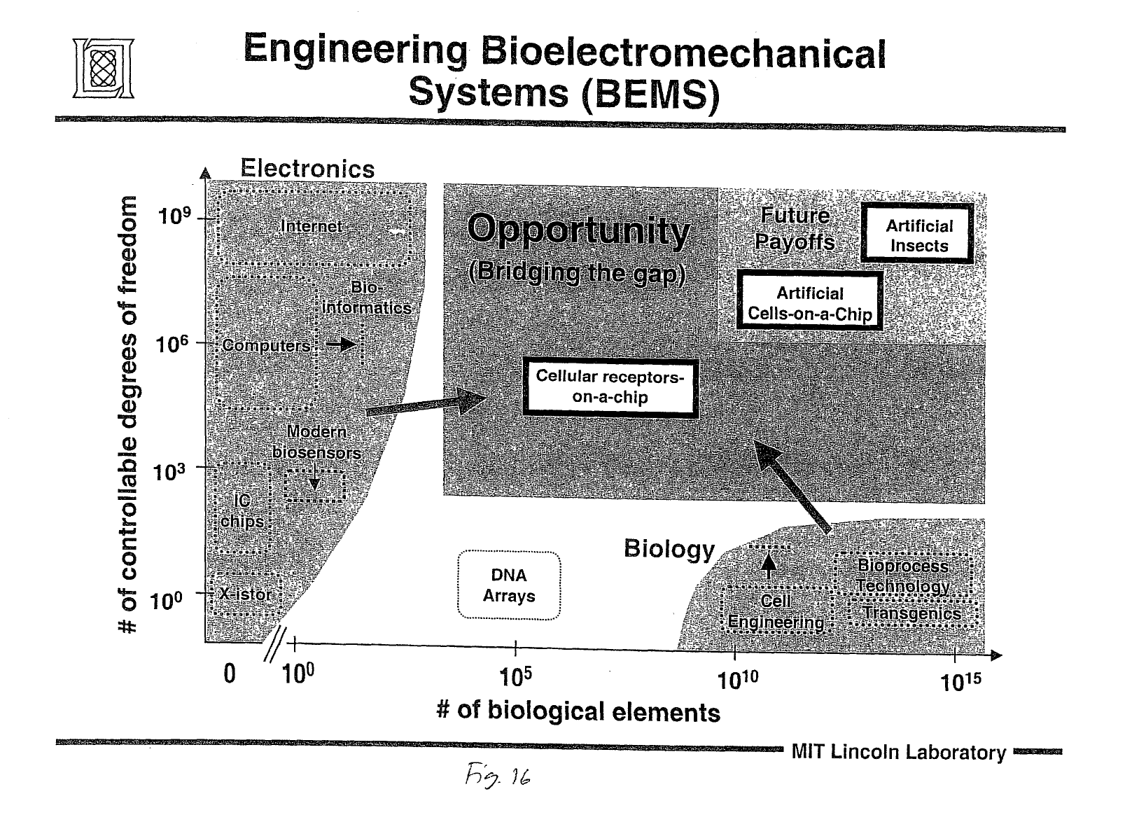

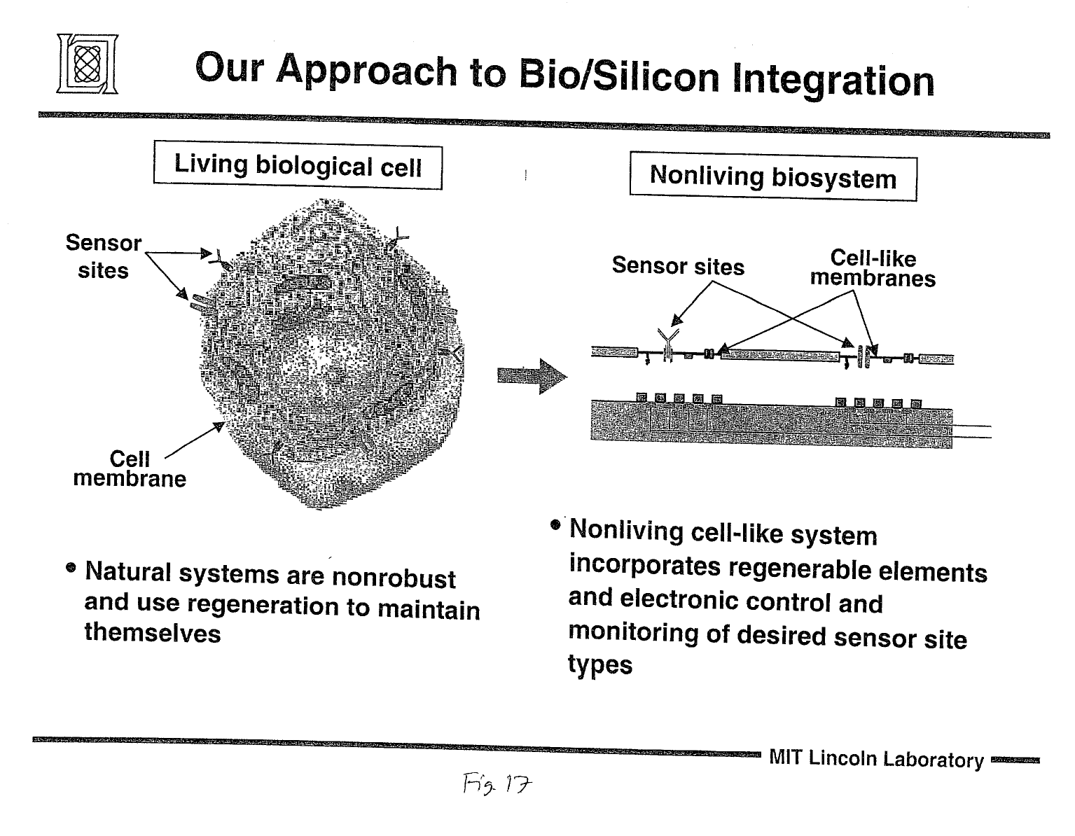

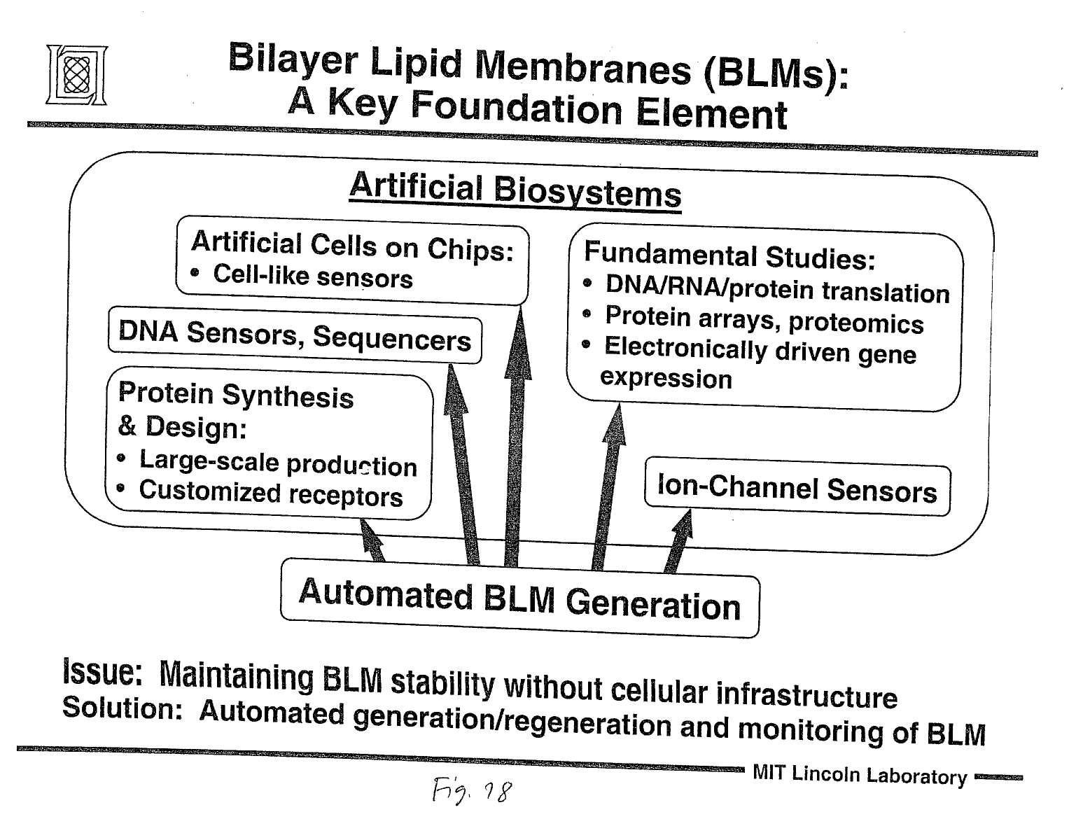

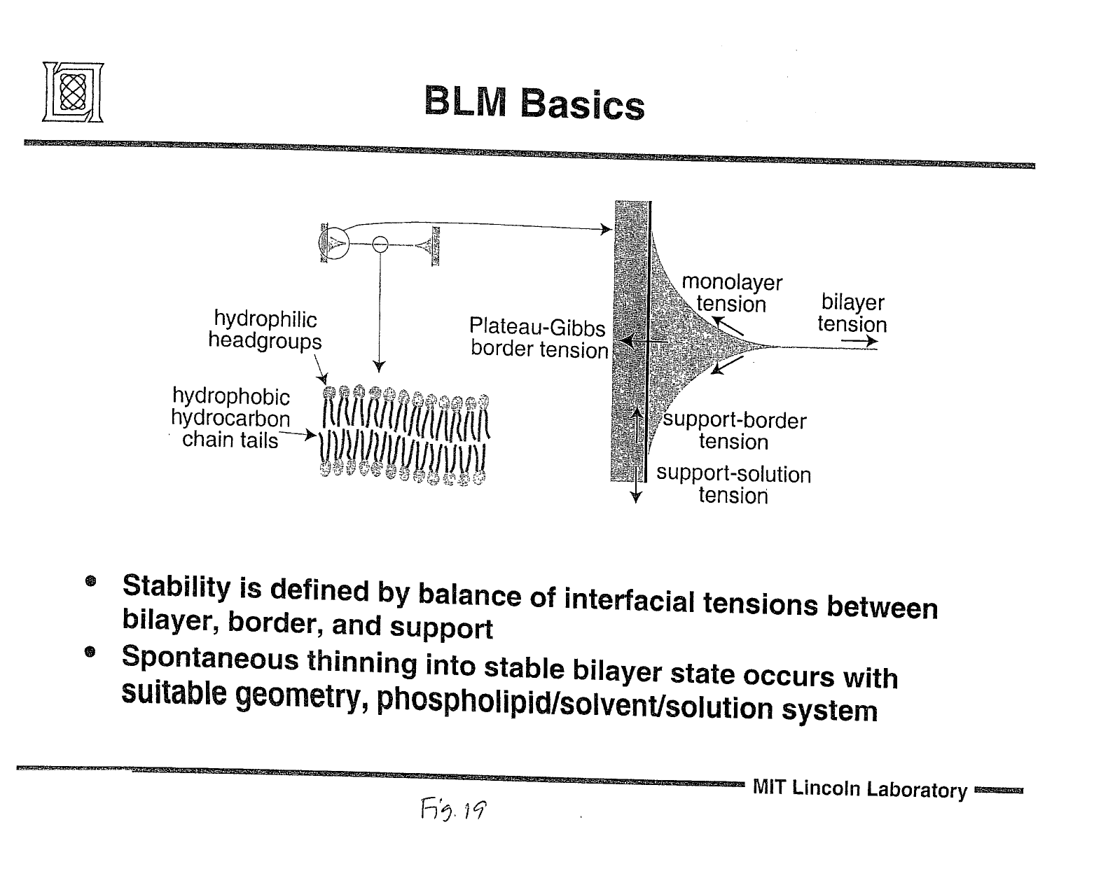

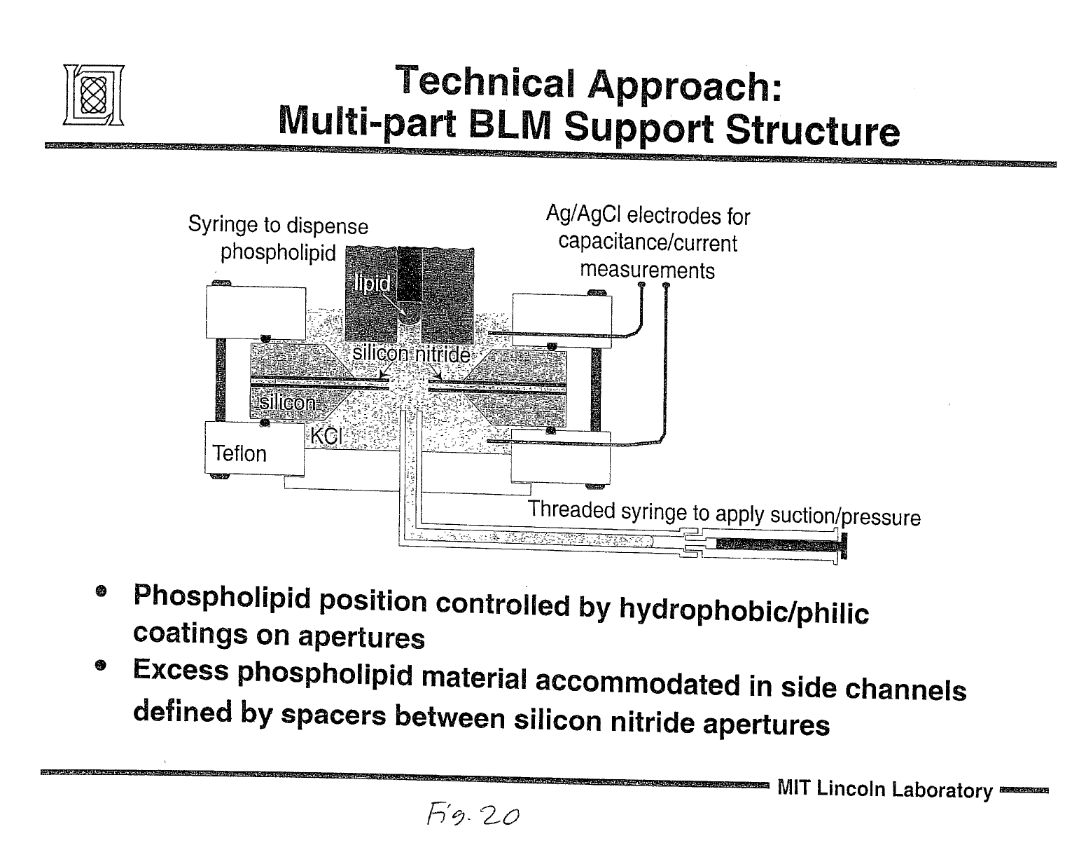

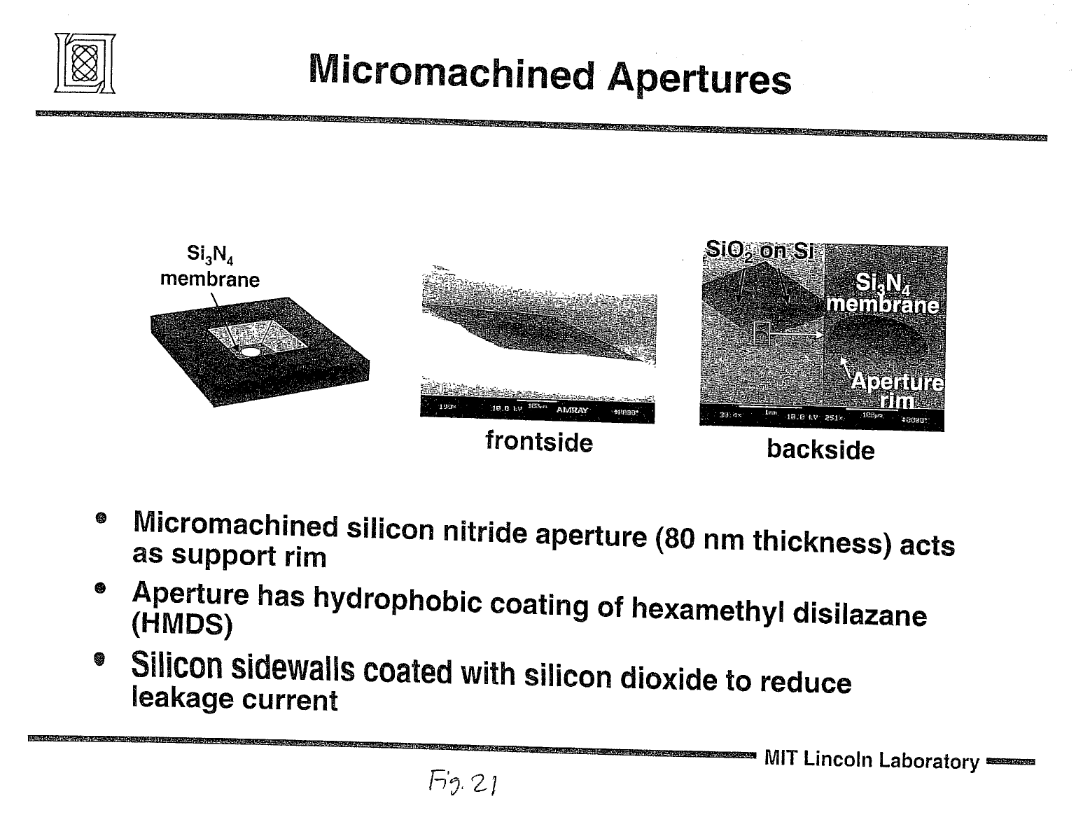

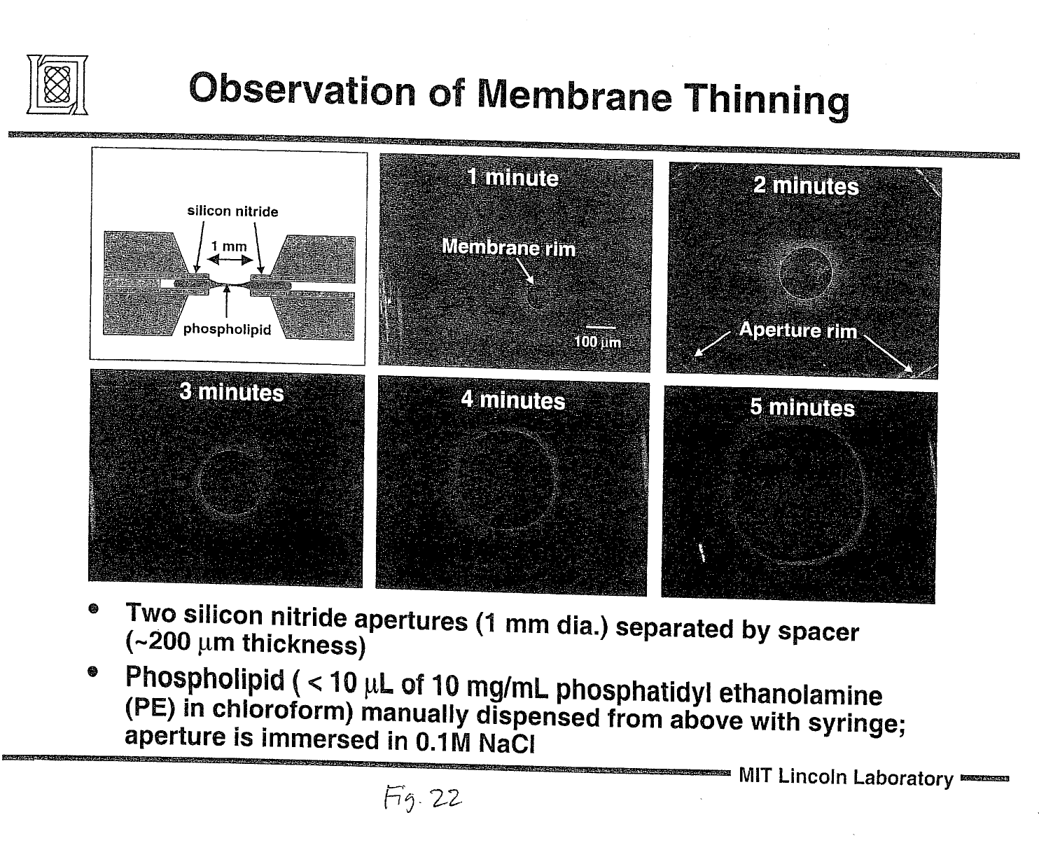

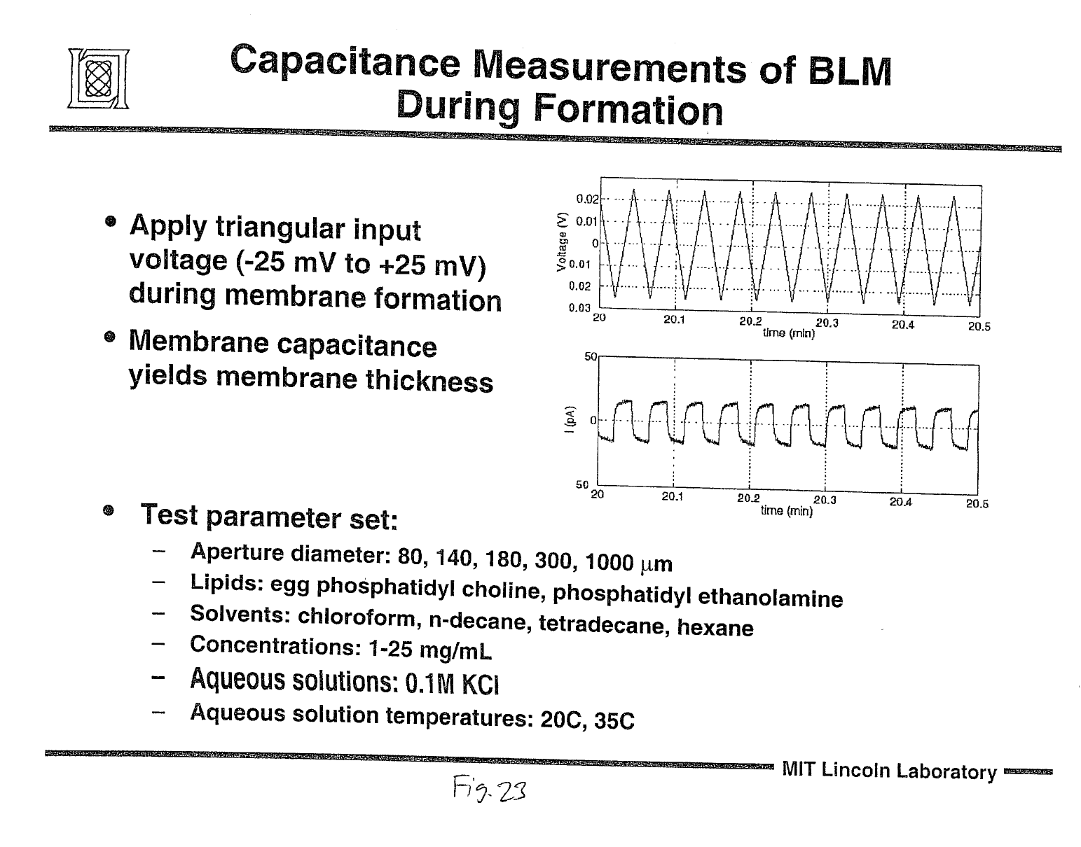

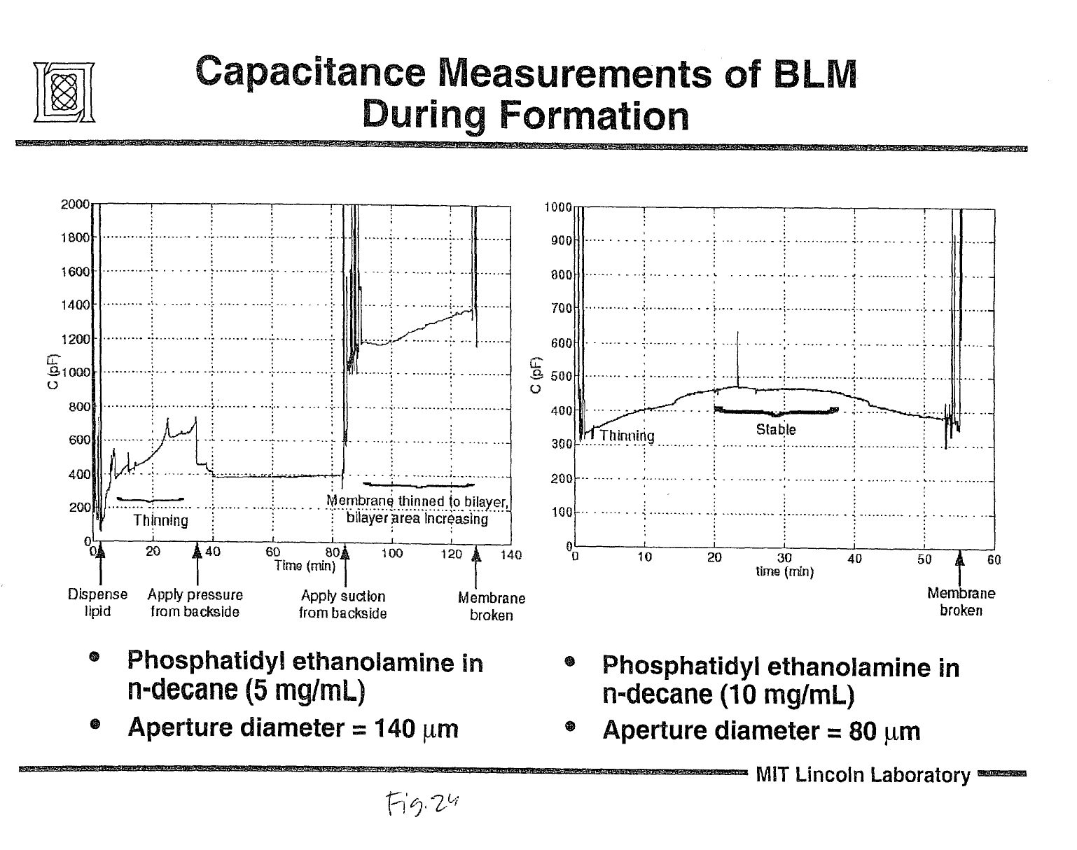

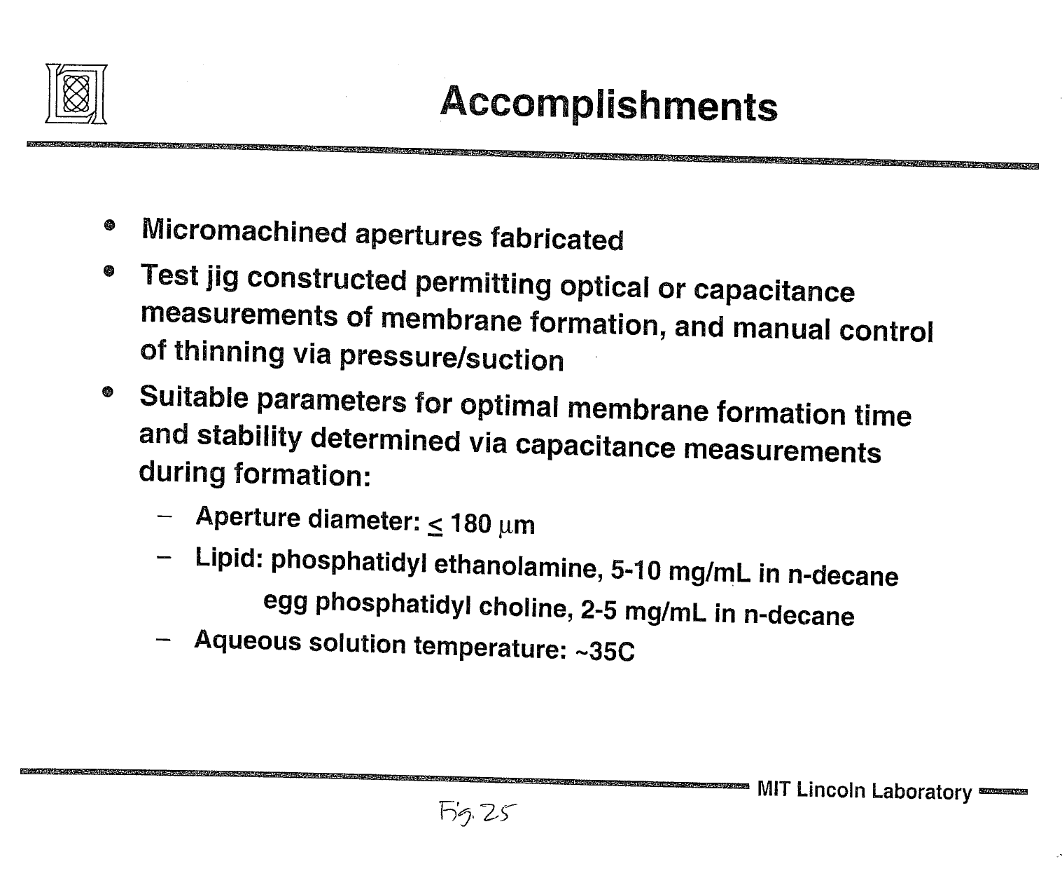

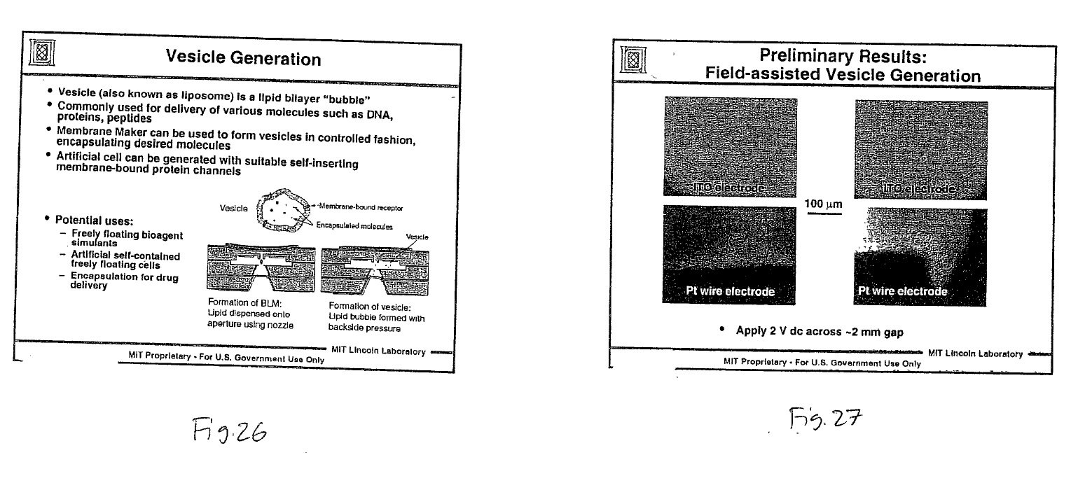

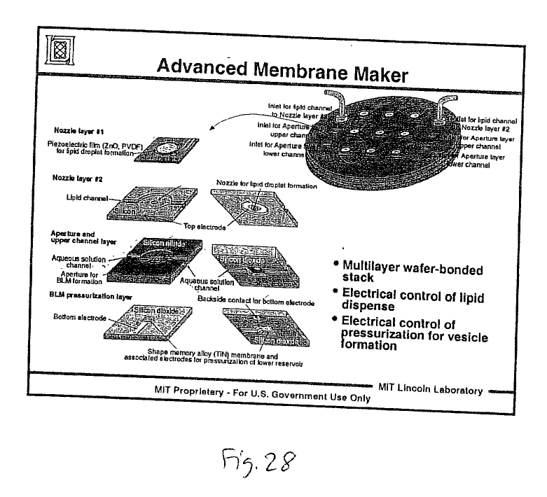

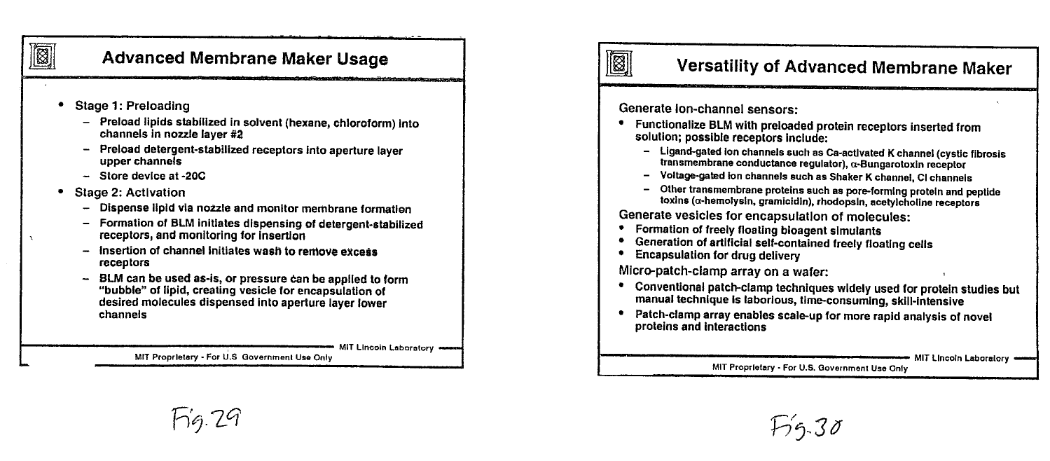

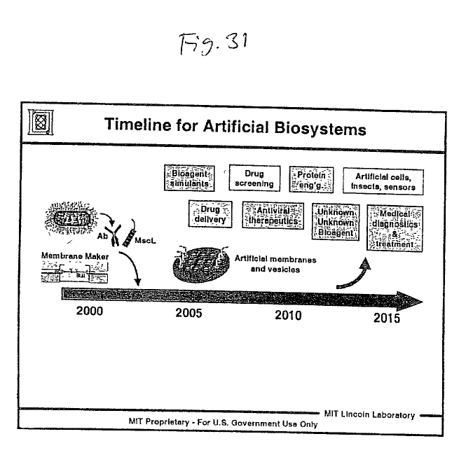

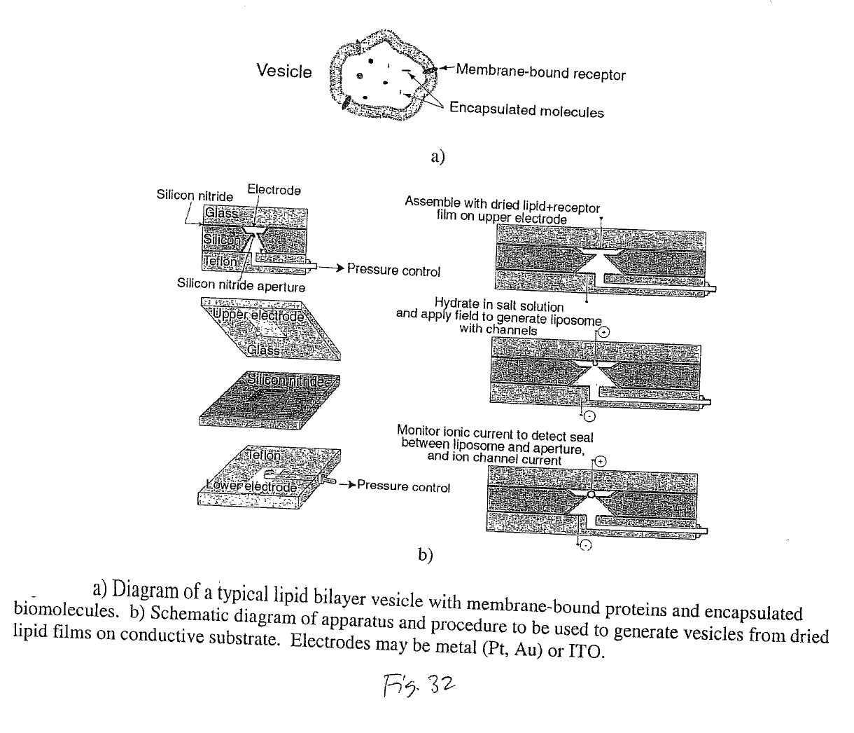

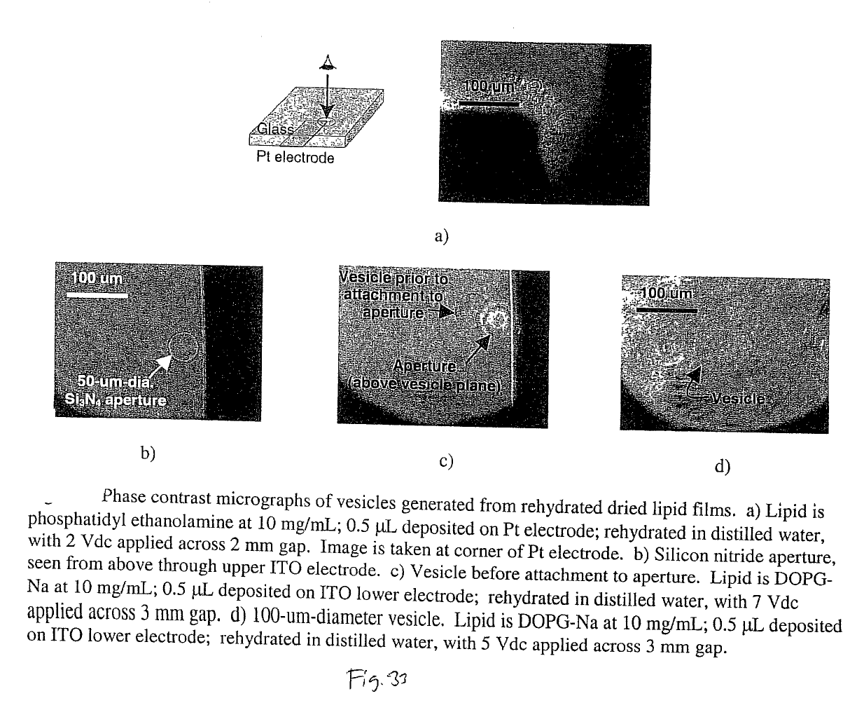

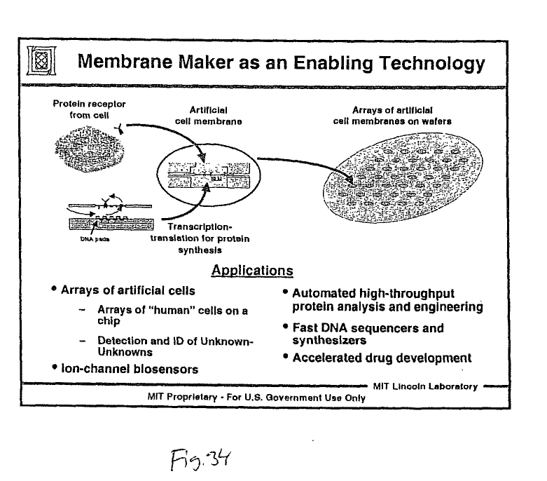

While this invention has been particularly shown and described with reference to FIGS. 1-34 and to preferred embodiments thereof, it will be understood by those skilled in the art that various changes in form and details may be made therein without departing from the scope of the invention encompassed by the appended claims.

What is claimed is:

1. A method of producing an artificial cell membrane, the method comprising:
(a) dispensing a membrane lipid across an aperture; and
(b) applying suction at the sides of the aperture, thereby causing the membrane lipid to thin;
thereby producing an artificial cell membrane.

2. The method of claim 1, further comprising the step of determining the capacitance of the membrane, thereby measuring the thickness of the membrane.

3. The method of claim 2, wherein the capacitance is determined during the application of suction, and once the desired thickness of the membrane is achieved, the suction is eliminated, thereby producing an artificial cell membrane of a desired thickness.

4. The method of claim 1, wherein the membrane lipid is selected from the group consisting of: a naturally-derived membrane lipid, and a synthetic membrane lipid.

5. The method of claim 1, wherein the membrane lipid is selected from the group consisting of: a phosphatidyl choline, a phosphatidyl ethanolamine, a phosphatidyl glycerol, a phosphatidyl serine, a phosphatidyl inositol, and a sphingomyelin.

6. The method of claim 1, wherein the membrane lipid is a mixture of different membrane lipids.

7. The method of claim 6, wherein the membrane lipid is a mixture of phosphatidyl choline and phosphatidyl ethanolamine.

8. The method of claim 1, wherein an electrical current is applied across the aperture during membrane formation, thereby causing pores to develop in the membrane.

9. The method of claim 1, wherein components of cell membranes are included in the membrane lipid.

10. The method of claim 9, wherein the components are selected from the group consisting of: receptor proteins, transport proteins, ion channel proteins, antibody receptor proteins, and signaling proteins.

11. The method of claim 1, where the membrane lipid is in the form of a liposome when it is dispensed across the aperture.

12. A method of producing a membrane, the method comprising:
(a) dispensing a polymer in liquid form across an aperture;
(b) applying suction at the sides of the aperture, thereby causing the polymer in liquid form to thin; and
(c) before, during, or after steps (a) and (b), applying heat to the polymer;
thereby producing a membrane.

13. The method of claim 12, further comprising the step of measuring the capacitance of the membrane, thereby measuring the thickness of the membrane.

14. The method of claim 13, wherein the capacitance is determined during the application of suction, and once the desired thickness of the membrane is achieved, the suction is stabilized, thereby producing a membrane of a desired thickness.

15. The method of claim 12, wherein the method further comprises heating the polymer prior to the polymer being dispensed across the aperture.

16. The method of claim 15, wherein the polymer is a thermoplastic.

17. The method of claim 12, wherein an electrical current is applied across the aperture during membrane formation, thereby causing pores to develop in the membrane.

18. An apparatus for producing an artificial cell membrane, the apparatus comprising:
(a) a member with at least one aperture; and
(b) at least one side channel adjacent to the aperture, through which suction can be applied across the aperture;
where, when a membrane lipid is applied across the aperture, an artificial cell membrane is formed.

19. The apparatus of claim 18, further comprising at least one electrode laterally adjacent to the aperture.

20. The apparatus of claim 18, further comprising two electrodes laterally adjacent to the aperture.

21. The apparatus of claim 20, further comprising an instrument for determining the capacitance between the electrodes.

22. An apparatus for producing a membrane, the apparatus comprising:
(a) a member with at least one aperture; and
(b) at least one side channel adjacent to the aperture, by which suction can be applied across the aperture;
where, when a polymer in liquid form is applied across the aperture, a membrane is formed.

23. The apparatus of claim 22, further comprising two electrodes laterally adjacent to the aperture.

24. The apparatus of claim 23, further comprising an instrument for determining the capacitance between the electrodes.

25. The apparatus of claim 22, wherein the polymer is a thermoplastic.
